(19) United States
(12) Patent Application Publication
(10) Pub. No.: US 2016/0262664 A1
(43) Pub. Date: Sep. 15, 2016

(54) Detection Of Disease Using Gesture Writing Bio-Markers

(71) Applicant: Michael Linderman, Ogdensburg, NY (US)

(72) Inventor: Michael Linderman, Ogdensburg, NY (US)

(21) Appl. No.: 15/067,169

(22) Filed: Mar. 10, 2016

Related U.S. Application Data
(60) Provisional application No. 62/131,180, filed on Mar. 10, 2015.

(51) Int. Cl. A61B 5/11; A61B 5/00; A61B 5/0488

(52) U.S. Cl. CPC A61B 5/1124; A61B 5/0488; A61B 5/4058; A61B 5/7246; A61B 5/4082; A61B 5/6806; A61B 5/6898

(57) ABSTRACT

A method, system and apparatus for detecting a disorder of the central nervous system, comprises: recording an EMG from muscle groups on left and right hands of patient; separating a gesture writing action into time intervals of postural and writing activity; recording EMG activity from the same muscle groups during a resting time period; determining a plurality of indicators, comprising: identifying tremor peaks in EMG spectral density; determining muscle tone like stiffness from EMG spectral density determining muscle weakness from EMG intensities; determining patterns of correlation waves; determining a delay from when a subject started writing after hearing an audio signal; determining the time of writing activity, when a pen is touching a tablet; determining the heights and shapes of characters from pen traces on a tablet; and assigning a point value to every indicator and calculating a combined score based on the point values.

### CROSS-REFERENCES TO RELATED APPLICATIONS

This application claims priority from U.S. Provisional Application Ser. No. 62/131,180 filed Mar. 10, 2015, which is hereby incorporated herein by reference in its entirety.

### TECHNICAL FIELD

This invention relates to a method, system and apparatus for detection of diseases of the nervous system using gesture writing bio-markers as revealed by EMG.

### BACKGROUND OF THE INVENTION

A method, system, and a device to diagnose neurodegenerative diseases or neurological disorders is described herein. This technology is used for pre-clinical diagnostics, monitoring of a disease, and evaluating the efficacy of a drug. It records electromyography from intrinsic hand muscles of two hands during gesture writing activity and rest. The same technology combines electromyography and analytical data from a computerized tablet for identifying the status of a disease. In the past, electromyography, handwriting traces and kinematics were used to study the status of the nervous system. These methods were limited as they worked in isolation and only provided the indirect analysis of nervous system functionality. Intuitively, bio-medical researchers came to the realization that a single bio-marker for diagnostics and monitoring is not good enough. The prevailing opinion in the biomedical community is that various approaches are needed; a single bio marker cannot be completely reliable and accurate diagnostics requires years of observation and the study of the medical history.

At this time there is no one solution on the market and clinicians rely on medical history and repetitive examination. The main reason for not having one diagnostic approach is the lack of understanding of the mechanisms of neurodegenerative and neurological disorders. On the other hand, our understanding of brain anatomy and functionality is considerably improved over the last years due to the latest advances in Deep Brain Stimulation (DBS) and neuro imaging experience. The goal of the present invention is to develop a noninvasive technology that takes into account a number of indicators. This technology analyzes motor neuronal activity and at the same time functioning in a paradigm of not handwriting, but rather “gesture writing”. This will allow combining the data from the EMG and kinematics in a controlled environment during the activation of very sensitive psychological network.

The problem of pre-clinical diagnostic and subsequent monitoring of aging and neurological disorders is solved by the present invention by presenting the gesture writing process as a combination of two activities, postural and writing, and combining them with resting time intervals. Resting is the time period when your arms and hands are resting on a desk while you are seated. Postural activity occurs in EMG, when a subject is holding a pen, but does not write on a tablet. In other words, a pen does not interact with the tablet. These type of gestures happen when a subject is about to write, just finished writing, or in the middle of writing on a tablet or any surface. This novel view on EMG activity recorded from intrinsic hand muscles in three different phases allows for much closer view on changes in neuronal functionality during a disease. Therefore, the indicators, such as tremor, stiffness, and weakness can be obtained in three time periods during one simple, economical, and noninvasive process. During the same session, one can measure the information about slowness, weakness, micrographia, loss of facial expression, memory and cognitive disorders, and the ability to sustain repetitive movement. Pearson time dependent correlations calculated from hand muscle activities during different phases of gesture writing were used in the past for the analysis of handwriting EMG. This time we included the “wavy” patterns in correlation functions as indicators of neurological disorders during gesture writing. These waves can appear during rest, postural, and writing activities. After determining the values of all indicators by assigning the appropriate weights to each indicator, we can evaluate a subject on the presents of a disease or on the status of a disease.

### BRIEF SUMMARY OF EMBODIMENTS OF THE INVENTION

According to one embodiment of the invention, a method of detecting a disorder of the central nervous system, comprises: recording an EMG from muscle groups on left and right hands of patient; separating a gesture writing action into time intervals of postural and writing activity; recording EMG activity from the same muscle groups during a resting time period; determining a plurality of indicators, comprising: identifying tremor peaks in EMG spectral density from more than one muscle group in each hand using separate time intervals; determining muscle tone like stiffness from EMG spectral density and in more than one muscle group using the separate time intervals; determining muscle weakness from EMG intensities in each time phase in more than one of the muscle group using the separate time intervals; determining patterns of correlation waves in more than one of the muscle groups using the separate time intervals; determining a delay when a subject started writing after hearing an audio signal; determining the time of writing activity, when a pen is touching a tablet; determining the heights and shapes of characters from pen traces on a tablet; and assigning a point value to every indicator and calculating a combined score based on the point values.

In a variant a method of detecting a disorder of the central nervous system, comprises: recording an EMG from muscle groups on left and right hands of a patient over time intervals, while the patient engages in writing activity; dividing the time intervals into periods of postural and writing activity; recording EMG activity from the same muscle groups during a resting time period; determining a plurality of indicators, comprising: identifying tremor peaks in EMG spectral density from more than one muscle groups in each hand using separate time intervals; determining muscle tone like stiffness from EMG spectral density and in more than one muscle groups using the separate time intervals; determining muscle weakness from EMG intensities in each time phase in more than one of the muscle groups using the separate time intervals; determining patterns of correlation waves in more than one of the muscle groups using the separate time intervals; and assigning a point value to every indicator and calculating a score based on the point values.

In another variant of the method of detecting a disorder of the central nervous system, the EMG is measured from two channels in each hand.

In a further variant of the method of detecting a disorder of the central nervous system, the disorder is Parkinson's disease.

In still another variant, a method of detecting a disorder of the central nervous system, comprises: recording an EMG from muscle groups on left and right hands of a patient over time intervals, while the patient engages in writing activity; dividing the time intervals into periods of postural and writing activity; recording EMG activity from the same muscle groups during a resting time period; wherein during the writing activity or rest, the disorder exhibits any of: tremor in each of the muscle groups at rest or in postural, stiffness in any of the muscle groups of any hand during postural or at rest, and weakness in akinesia or bradykinesia.

Other features and aspects of the invention will become apparent from the following detailed description, taken in conjunction with the accompanying drawings, which illustrate, by way of example, the features in accordance with embodiments of the invention. The summary is not intended to limit the scope of the invention, which is defined solely by the claims attached hereto.

### BRIEF DESCRIPTION OF THE DRAWINGS

The present invention, in accordance with one or more various embodiments, is described in detail with reference to the following figures. The drawings are provided for purposes of illustration only and merely depict typical or example embodiments of the invention. These drawings are provided to facilitate the reader's understanding of the invention and shall not be considered limiting of the breadth, scope, or applicability of the invention. It should be noted that for clarity and ease of illustration these drawings are not necessarily made to scale.

Some of the figures included herein illustrate various embodiments of the invention from different viewing angles. Although the accompanying descriptive text may refer to such views as “top,” “bottom” or “side” views, such references are merely descriptive and do not imply or require that the invention be implemented or used in a particular spatial orientation unless explicitly stated otherwise.

FIG. 1 is a block diagram of preprocessing of EMG signals going to a Tablet PC.

FIG. 2 illustrates differential channels of EMG electrodes on both hands of a patient.

FIG. 3 illustrates a trial where tremor peaks are observed in the spectral density of EMG signals, where spectral density is derived using Fast Fourier Transform for each trial and averaged over all trials.

FIGS. 4a, 5a, 6a, 7a, and 8a illustrate EMG measurements before administration of medicine for, respectively, stiffness during postural, weakness, tremor during rest, stiffness during rest, and tremor during postural.

FIGS. 4b, 5b, 6b, 7b, and 8b illustrate EMG measurements after administration of medicine for, respectively, stiffness during postural, weakness, tremor during rest, stiffness during rest, and tremor during postural.

FIG. 9 is a graphical representation of the timing of the trials.

The figures are not intended to be exhaustive or to limit the invention to the precise form disclosed. It should be understood that the invention can be practiced with modification and alteration, and that the invention be limited only by the claims and the equivalents thereof.

### DETAILED DESCRIPTION OF THE EMBODIMENTS OF THE INVENTION

From time-to-time, the present invention is described herein in terms of example environments. Description in terms of these environments is provided to allow the various features and embodiments of the invention to be portrayed in the context of an exemplary application. After reading this description, it will become apparent to one of ordinary skill in the art how the invention can be implemented in different and alternative environments.

Unless defined otherwise, all technical and scientific terms used herein have the same meaning as is commonly understood by one of ordinary skill in the art to which this invention belongs. All patents, applications, published applications and other publications referred to herein are incorporated by reference in their entirety. If a definition set forth in this section is contrary to or otherwise inconsistent with a definition set forth in applications, published applications and other publications that are herein incorporated by reference, the definition set forth in this document prevails over the definition that is incorporated herein by reference.

An integrated technology for diagnosing diseases is presented that consists of an apparatus that a person wears on two hands whereby the electromyography (EMG) is recorded from hand muscles during rest and handwriting. When an EMG is recorded during handwriting, it is synchronized with the data from a tablet, when a pen is in contact with the tablet.

This technology combines measurements of tremor, stiffness, slowness, balance issues, etc. The obtained EMG signals are analyzed and the presents of Parkinson's disease (PD) are determined. The same technology can be used for the monitoring of the state of PD and the effects of medication. In this case the magnitudes of each end point over time have to be compared, or before and after taking medication.

Technology for detecting Parkinson's disease (PD) comprises of a group of indicators (endpoints) that are based on various Electromyography (EMG) properties of hand muscles during gesture writing as well as kinematics and pen traces. Gesture writing is the type of writing, when part of the time a pen is touching the tablet (writing activity) and part of the time a pen is not touching the tablet (postural activity). During postural period a hand is held against gravity. Postural EMG activity can be found programmatically by analyzing the writing activity, since it is known from the tablet recording when a pen was touching a tablet and not touching the tablet. In addition, EMG at rest is recorded, while both hands are lying on a table. The properties of resting EMG signals are also included in the analysis. Two differential channels of EMG are recorded from each hand. EMG recordings are synchronized with the input from the tablet PC.

PD patients often have the resting tremor that is suppressed in activity, such as handwriting. Therefore, EMG during three periods is analyzed: rest, postural, and writing activity.

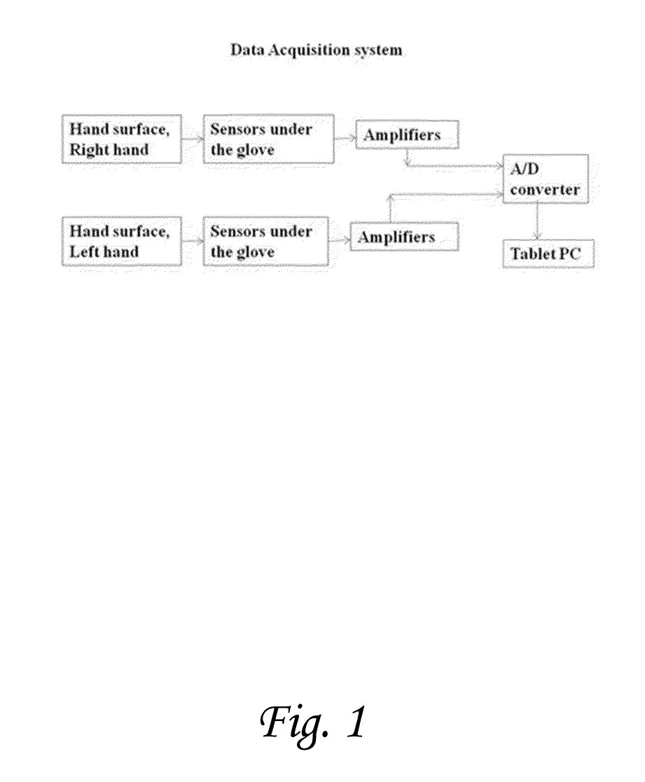

Referring to FIG. 1, a system and method for detecting disorders of nervous system is described. The system comprises the following components: 1) A system for preprocessing of Electromyography (EMG) signals; 2) A tablet PC system that includes a computerized device and a tablet; 3) Computer readable instructions for acquiring EMG signals, pen traces, and temporal events indicating the activity of the pen; 4) Computer readable instructions for analyzing the resulting data containing synchronized EMG and data from the tablet.

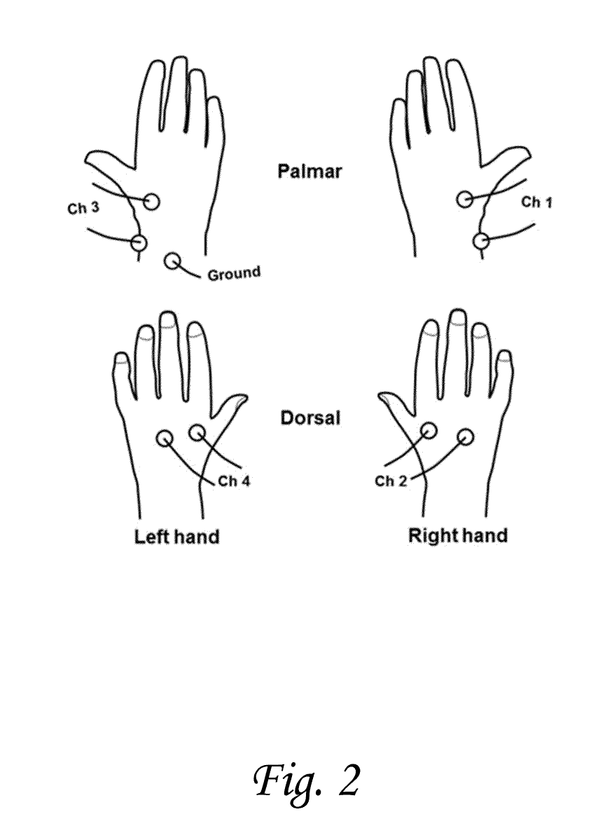

The system for preprocessing EMG signals is connected to the computerized device and a tablet as shown in FIG. 1. The connection between A/D converter may be wired or wireless. In the case of a wireless connection, each hand has its own A/D converter. The A/D converter may include a processor, USB connection, or a modem. In order to record EMG signals from hands, suitable commercially available electrodes are used. FIG. 2 illustrates placements of differential electrodes on right and left hands.

For tablet to EMG Data Synchronization, in one example, a Samsung Tablet PC was used like a mouse in the system and therefore handled through the Microsoft Windows event queue. Every time the pen moves is pressed down or lifted up an event is sent to LabVIEW with a 32 bit millisecond “tick count” that tells LabVIEW when the event happened. The EMG data is recorded using a National Instrument DAQ device (NI 6008). The data received from the DAQ board (“digitizer”) is marked with a 128 bit timestamp, which is different and not directly related to the “tick count”.

To synchronize the data from the two different sources, relative time is recorded, i.e. the “tick count” and the timestamp of the point when the user presses the ‘Start Recording’ button are recorded and subtracted from all subsequent recorded “tick counts” and timestamps. In this way when the ‘Start Recording’ button is pressed, the zero point in time for the recording is set. The table below shows various tick counter values that correspond to various Tablet actions.

TABLE 1
Tablet File Event
Types:

| | |
|---|---|
| Start Recording | 1 |
| Stop Recording | 0.5 |
| Press-Down | 0 |
| Move | 0 |
| Lift-Up | 2 |
| EEG Sync Pulse Start | 5 |

EMG data is recorded with the computer clock data. In order to line up the times, the Get Timestamp vi function gets the current tick counter value and the current time at the same time. It generates the clock value of the start recording event as follows:

Clock start=Clock current−(Tick current−Tick start)

In the EMG and Tablet files time is recorded as elapsed time in seconds since Start Recording Event. Lift-up event in tablet file is marked with number 2 in Event Type column. The time of the row in the tablet file can be used to find corresponding EMG value in EMG file. The trial counter is updated when the test subject lifts the pen for longer than the time specified in Idle before next trial (ms) control field. When recording words with letters that do not require lift-ups, each lift-up event corresponds to the end of a letter. When characters are continuously written, lift-up code denotes end of character. The trial is advanced. The tablet PC generates the beeping sound in the end of each trial, which prompted a subject to start writing.

In one example, 100 trials were administered. A beep sound denotes the end of one trial and beginning of the next trial. Writing activity was analyzed while a subject was writing a number “3” in cursive. The length of each trial was 5 sec. During “rest,” trials with a length of 5 sec each were generated. Postural trials were selected by a computer program. 100 trials for each type of activity were recorded. A large number of trials (around 100) is very important in the analysis, because muscle activity has high variability. A statistical ensemble is created in order for statistical analysis to be applied. A confidence interval was calculated to confirm the existence of peaks in Fast Fourier Transform (FFT).

Experimental Procedure

Recordings were made in three conditions in a fixed sequence: resting trials, character-writing trials alternated with segments where the pen was kept lifted by flexing the wrist (i.e., postural segments), and finally word-writing trials. The complete test was performed within 35 minutes including putting on and taking off the gloves.

Condition 1: Resting. The experimental session started with a recording where the participants were resting with both hands, forearms, and elbows on the table. During one continuous recording the experimenter triggered at least 25 trials of 5 seconds by touching the electronic pen on the tablet after each beep. In total 4 such files were recorded with 1-minute pauses in between, yielding 100 resting trials in total. During the pause the participants rested their hands on their knees. (EMG data was not recorded.) This prevented fatigue, helped blood circulation and noticeably reduced stiffness in the upper limbs.

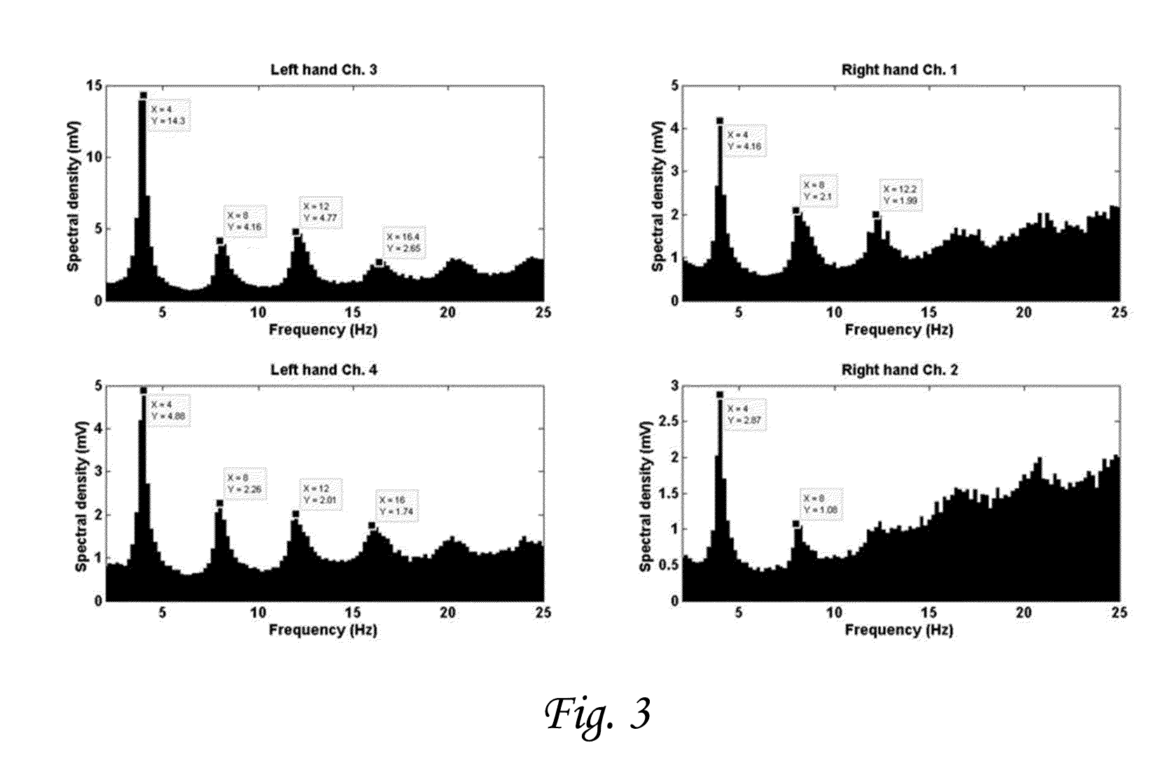

Condition 2: Character-Writing and postural control of pen lift. After the resting session the participants were instructed to wait with the pen lifted while the entire forearm including elbow rested on the table. Upon hearing the beep participants were to touch the tablet with the pen and write character “3” as fast and convenient as possible and then lift the pen until the next beep. The next beep was automatically generated 4 seconds after the pen lift. As the participants were instructed to keep their forearms resting on the table the thumb muscles were optimally involved in producing character “3”. The non-writing arm rested on the table as in the resting condition. The writing segment was defined as the 5-second segment starting 2 seconds before the pen touched the writing surface till 3 seconds after that (See FIG. 3). We also defined a postural segment starting when the pen was lifted till the beep that was generated 4 seconds later. Thus each trial included a writing segment and a postural segment. The experimenter ensured that after the first trial at least 25 good trials were recorded. The experimenter ensured that at least 25 successful trials were recorded. This means that the subject had to write a complete character and only after that they lift up the pen and waited for a beep to start writing another character. All trials, including incidentally failed trials (except first and last one) were included in the analysis. After recording 25 acceptable trials, the participant was instructed to pause for 1 minute with both hands on the knees like in the resting condition. In total 4 files of 5 minutes were recorded yielding 100 trials.

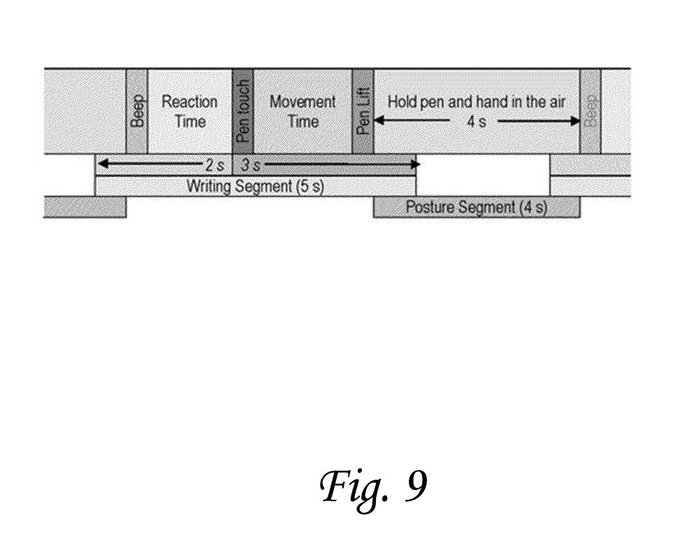

Referring to FIG. 9, a graphical representation of the timing of the trials is provided. The 5-second writing segment and the 4-second posture segment overlap. The reaction time and the movement time are around 1 second. The total recording block of at least 25 of these trials lasted about 5 minutes.

Condition 3: Word-Writing. This condition was similar to the character-writing condition except that the participants were requested to write the word “cow” in cursive and without pen lift instead of writing “3”. One block of trials was recorded.

Our Norconnect DH software (written in MATLAB, MathWorks, Natick, Mass., USA) processed the EMGs and handwriting kinematics to quantify 8 PD indicators (See Table 2). We estimated 3 of the 4 cardinal signs of Parkinson's disease: resting tremor, stiffness during rest, and akinesia (i.e., the slowness to begin a movement, as quantified by reaction time). The 4th sign of PD is postural instability but we did not attempt to quantify this sign as this rarely occurs in the early stages of PD. We estimated 5 additional indicators: tremor and stiffness during a postural activity (i.e., lifting the pen), muscle weakness during writing, bradykinesia (i.e., slowness of movement execution, as quantified by movement time), and micrographia (i.e., writing extremely small). Each indicator was based on 1 to 4 EMG or handwriting features. In total we measured 21 parameters in each test.

Table 2: Criteria used for each of the parameters. The Norconnect DH system measures 21 parameters of EMGs in left and right hands and the pen movements in the preferred hand during handwriting tasks, a postural task (i.e., keeping the pen lifted), and during rest. Channels 1 and 2 (Ch1, Ch2) represent the EMG of the right-hand thumb agonist and antagonist, resp., and Channels 3 and 4 (Ch3, Ch4) of the left hand. All participants were right handed. The measures are compared against criteria indicative of PD. In order to decide which of the 8 PD indicators receive a score of 1 or 0.5 if positive or 0 if negative. The compound score is the sum of scores except muscle weakness and micrographia are only added with weight 0.5 if the other indicators add up to at least 2. A compound score ≧3 is indicative for Parkinson's disease.

TABLE 2

| | | | | Score if |
|---|---|---|---|---|
| Symptom | Indicator | Parameters | Criterion | Positive |
| Tremor | 1. EMG during | 1. Ch1 | Significant spectral peak at 3-6 | 1 |
| | resting condition | 2. Ch2 | Hz in at least one of the 4 | |
| | (Cardinal sign 1/4) | 3. Ch3 | channels | |
| | | 4. Ch4 | | |
| | 2. EMG during | 5. Ch1 | | 1 |
| | postural segment | 6. Ch2 | | |
| | (Cardinal sign 4/4) | 7. Ch3 | | |
| | | 8. Ch4 | | |
| Stiffness | 3. EMG during | 9. Ch1 | Spectral density maximum >0.5 | 1 |
| | resting condition | 10. Ch2 | mV in at least one of the 4 | |
| | | 11. Ch3 | channel | |
| | | 12. Ch4 | | |
| | 4. EMG during | 13. Ch1 | Spectral density maximum >1 | 1 |
| | postural segment | 14. Ch2 | mV in at least one of the 2 | |
| | (Cardinal sign 2/4) | 15. Ch3 | channels of the writing hand | |
| | | 16. Ch4 | or >0.5 mV in at least one of | |
| | | | the 2 channels of the resting hand. | |
| Muscle Weakness | 5. EMG during | 17. Ch1 | Maximum intensity <0.005 (mV)² | 0.5 (*) |
| (Asthenia) | writing “3” | 18. Ch2 | in at least one of the 2 | |
| | | | channels of the writing hand | |
| Akinesia | 6. Pen kinematics | 19. Reaction | Interval between beep and first | 1 |
| (Movement initiation slowness) | during writing “3” | Time to | Pen Touch to write “3” >1 sec | |
| | (Cardinal sign 3/4) | write “3” | | |
| Bradykinesia | 7. Pen kinematics | 20. Movement | Interval between first and last | 1 |
| (Movement execution slowness) | during writing “3” | Time to | pen- touch movement during | |
| | | write “3” | writing “3” >1 sec | |
| Micrographia | 8. Pen kinematics | 21. Height | Difference between highest and | 0.5 (*) |
| (Diminishing handwriting) | during writing “cow” | of word | lowest Vertical Position <7 mm | |
| | | “cow” | | |

(*) Count weakness and micrographia (with score 0.5) only if the scores of the extended cardinal symptoms (resting and postural tremor, resting and postural stiffness, akinesia, and bradykinesia) add up to 2 or more.

A. EMG Tremor Peaks

One indicator is EMG tremor peaks and their suppression in Fast Fourier Transform (FFT) during rest, postural, and writing activities. Calculation of confidence interval and standard deviation is important for identifying the tremor peaks. A large number of trials (around 100) is important in an analysis, because the EMG has high variability. A statistical ensemble is crated to apply statistical analysis. A confidence interval is calculated to confirm the existence of peaks in Fast Fourier Transform (FFT).

Spectral Properties of EMG Signals

To examine the spectral properties of EMGs, using the Fast Fourier Transform, the spectrum (or spectral density) of the signals in each trial is computed, defined as the modulus of the complex Fourier harmonics, mn,α(f), where f is the frequency, while n and a enumerate trials and channels, respectively. However, owing to strong variability of signals from trial to trial, the spectrum also exhibits strong variations from trial to trial. Therefore, the spectral density is averaged over trails, Mα(f)=(1/N)Σtrials mn,α (f), where N is the total number of trials. The long trial duration (5,000 ms) allows the computation of the spectrum in steps of 0.2 Hz.

It should be also emphasized that in many cases the magnitude of higher-frequency peaks exceed the magnitude of the lowest-frequency peak with a central frequency of about 5 Hz. This differs from accelerometer measurements [2], which show very low magnitudes of the higher-frequency peaks compared to the lowest-frequency ones.

If the standard deviation (STD) of the spectral density from trial to trial is calculated, it is easily seen that STD is of the same order of the magnitude as the spectral density itself. This means that owing to very high variability of harmonics in the peaks from trial to trial, the peaks cannot be observed in individual trials. To find a confidence interval (with the confidence level of 95%), one observes distribution of the spectral density values, included in the peaks, and finds that the probability density function is well fitted by the normal (Gaussian) probability distribution function. Therefore, the upper and lower limits of the confidence interval, CI±, can be computed as,

$${CI}_{\pm } ( f ) = 1.96 \times \frac{STD ( f )}{\sqrt{N}} \qquad ( 1 )$$

where STD is the standard deviation, and N is the number of trials. Where STD is the standard deviation, and N is the number of trials. Since the limits of the confidence interval weakly depend on the number of trials (as N−1/2), for practical purposes the number of trials can be decreased to 100-200, preserving statistical significance of the results.

Sometimes the peaks are observed for patients with visible shaking of the corresponding hand, but sometimes shaking was not observed. Moreover, one may observe EMG spectral density peaks in the hand muscles, which are not engaged in generation of hand movements.

Therefore, in contrast to conventional definitions of tremor “tremor” herein is defined as an appearance of peaks in the spectral density of EMG signals. This appears to allow more sensitive observation of disease progression and establish earlier diagnosis. Tremor is determined by the presence of FFT peaks in intrinsic hand muscle groups.

The definition of tremor in this document helps to better detect and identify it. In the technology presented herein, tremor is represented by characteristic low frequency EMG peaks in FFT spectrums as opposed to observed shaking in subject's hands. This is not different from a clinical description and only helps to detect and quantify tremor better. Referring to FIG. 3, tremor peaks are observed in the spectral density of EMG signals, where spectral density is derived using Fast Fourier Transform for each trial and averaged over all trials.

B. Stiffness

EMG during rest and postural activities contains a characteristic component of stiffness, which is another indicator of PD. This component was calculated as a magnitude of spectral density between 2-400 Hz, for example, as an amplitude or a mean value of spectral density. It is important to note that all previous researchers and clinicians were evaluating stiffness, rigidity, and even muscle tone as a resistance to movement. In contrast, the present invention evaluates the component of stiffness during a non-movement state or activity. A live muscle is always receiving and sending signals. The present invention considers the high muscle activity at rest or postural as abnormal. Also confirmed through research, is this characteristic that is calculated, correlates very well with the results of clinical exams and the feeling of subjects regarding to their stiffness. After the postural density is calculated from EMG using Fourier analysis, it needs to determined, if the maximum spectral density in mV (determined in any EMG channel) exceeds the amount for control subjects which do not have Parkinson's disease and in the same time do not have other muscle diseases affecting their muscle tone like dystonia. Many programming environments would have the functionality to calculate these types of maximums and will allow one to exclude the maximums of erroneous peaks like electrical interference, e.g. max (EMG_spectrum) in MatLab®. It is assumed that if the muscle activity is higher in a test or patient subject than in control subjects at rest and postural, then it is an indication of stiffness. Postural activity, when a subject is holding a hand with the pen against gravity, should have the higher level of EMG spectral density in mV, than the spectral density of muscles at rest. Therefore the maximum of spectral density at rest and postural for control subjects should be determined separately and compared to the potential PD patients.

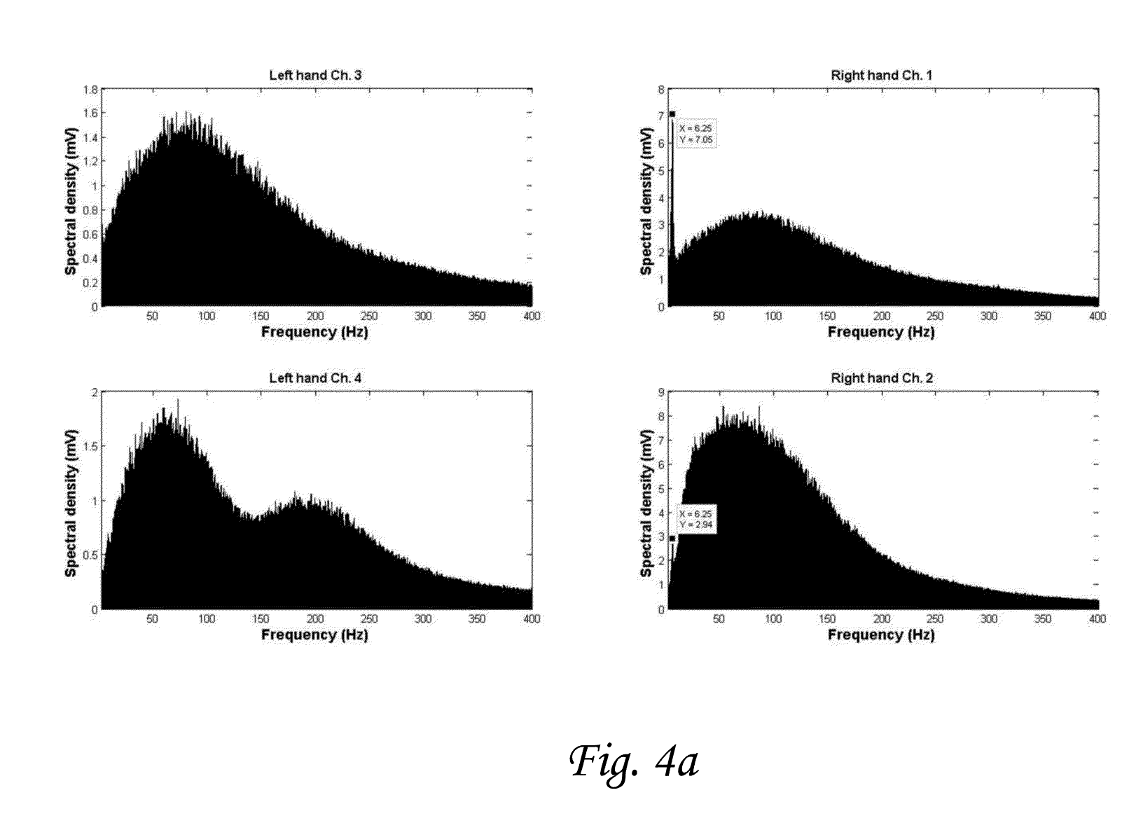

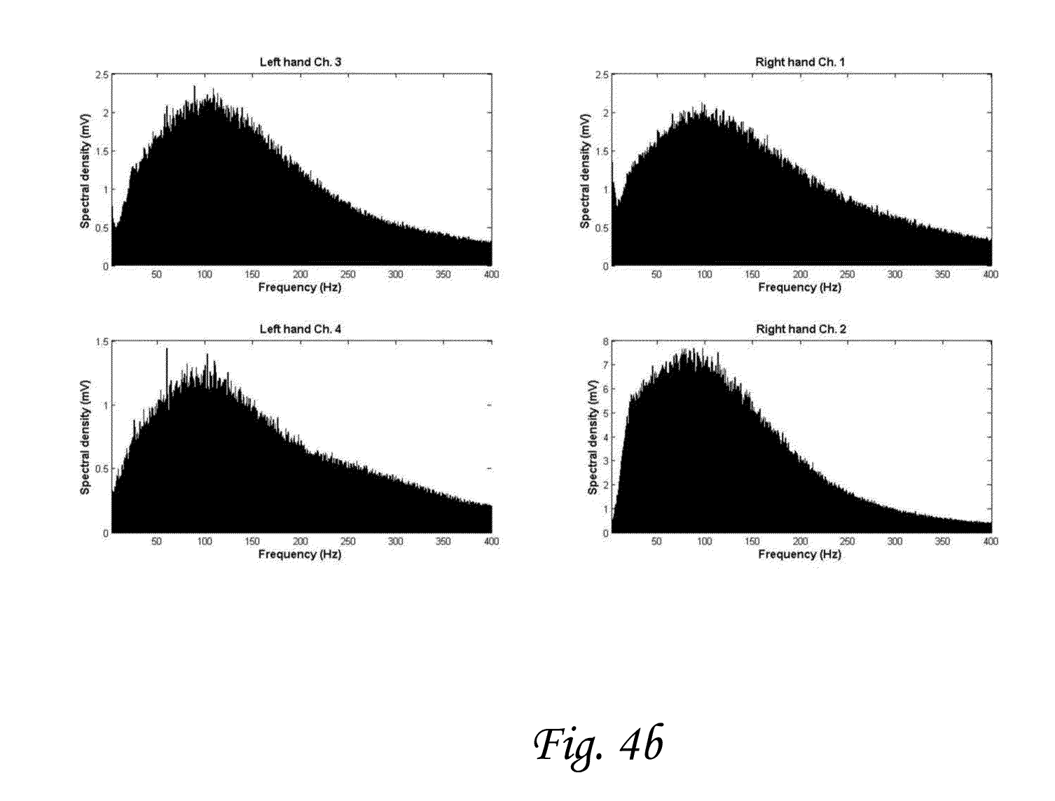

Referring to FIG. 4, stiffness is observed in spectral density of EMG signals, where spectral density is derived using Fast Fourier Transform for each trial and averaged over all trials. After obtaining the data for spectral density, the mean value of spectral density in each channel of EMG is calculated. Slowness at gesture writing that is expressed as akinesia (reaction time on the sound of the beep) and bradykinesia (writing time). A beep sound is used, that marks the end of one trial and beginning of next. The time when a pen was connected to paper is also used.

C. Abnormality in Time Dependent Correlation Functions

Correlations functions were defined in the following publication: Rupasov V I, Lebedev M A, Erlichman J S, Lee S L, Leiter J C, Linderman M (2012) Time-dependent statistical and correlation properties of neural signals during handwriting, PLoS ONE 7(9): e43945. Abnormality in said time dependent correlations during rest, postural, and writing activities. Here one can visually detect this abnormality in time dependent correlations, when one observes a wave pattern in correlations. Correlations functions are calculated in each channel and between channels. One looks for the pattern as a potential indicator of an abnormality related to movement disorders.

D. Weakness During Gesture Writing

Weakness during gesture writing may correspond to Parkinson's disease (PD). The weakness in different EMG channels (groups of muscles) is determined by the intensities of EMG signals during writing a character. We consider muscles to be weak, if the intensity is less than 0.05 (mV)2.

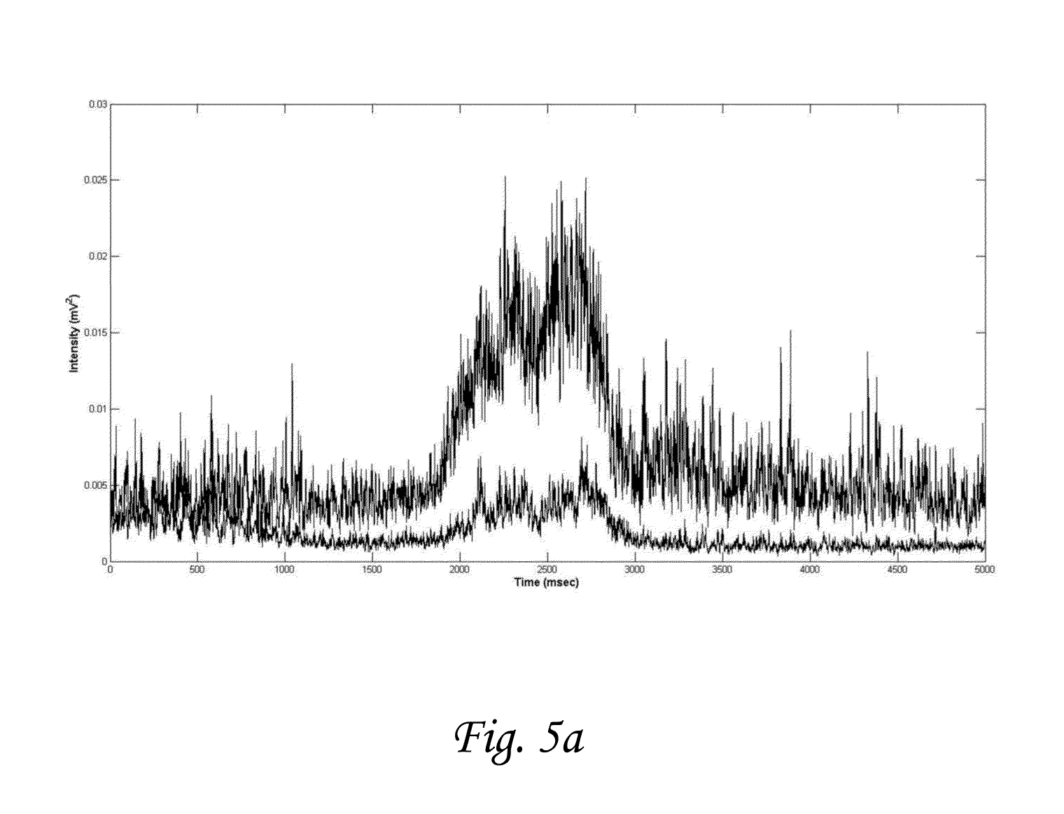

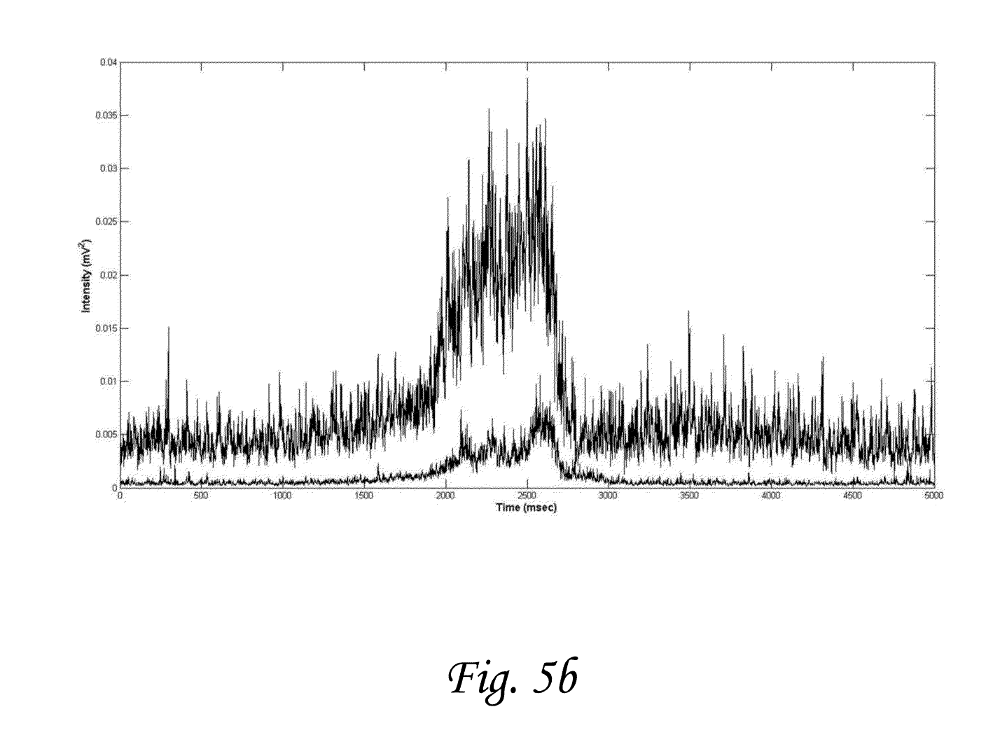

Referring to FIG. 5a, weakness is detected in EMG signals during writing, averaged over 100 trials.

E. Micrographia

Micrographia is determined from writing words like “cow” in cursive. One type of micrographia is related to the small height of characters written with some type of time interval, e.g. 5 sec. In this case the heights of characters are smaller and smaller, as opposed to the same heights of characters written individually.

In order to detect PD in accordance with the principles of the invention, a score is computed based on the above indicators which have to be measured. Any one of the above indicators measured alone, is not conclusive of PD. One can conclusively diagnose a subject with PD, only when the following criteria are applied. A list of the primary symptoms comprises: tremor (rest and/or postural in any of 4 channels); stiffness (rest and/or postural in any of 4 channels); slowness in reaction time and writing time. Weights are assigned to the symptoms referred to as endpoints. When a primary symptom is present, its value is 1. When a symptom is absent, its value is 0. When correlation abnormalities represented as a wave like pattern at rest and/or postural are present, they are also counted as 1. At least two of the primary symptoms (1A: rest tremor, 1B: postural tremor; 2A: rest stiffness, 2B: postural stiffness; 2C: reaction time, 2D: writing time) and/or correlation abnormalities have to be present in order to add secondary symptoms such as weakness and micrographia, if they are present. In other words, When these secondary symptoms are present, they are assigned value 0.5. The combined score is determined by assigning the appropriate points to the following indicators:
rest tremor 1 or 0
postural tremor 1 or 0
rest stiffness 1
postural stiffness 1 or 0
akinesia 1 or 0
bradykinesia 1 or 0
rest waves 1 or 0
postural waves 1 or 0
Weakness 0.5 or 0, if 2 points from rest or postural tremor, rest or postural stiffness, akinesia, bradikinesia.
Micrographia 0.5 or 0, if 2 points from rest or postural tremor, rest or postural stiffness, akinesia, bradikinesia.

A combined score is computed by adding all the assigned point. PD is diagnosed when the combined score is greater than or equal to 3.

The following are examples of calculating the combined score.

TABLE 3
Example measurement and calculation of 3 subjects.

| | Subject1 | Subject2 | Subject3 |
|---|---|---|---|
| rest tremor 1 | 0 | 0 | 1 |
| postural tremor 1 | 0 | 0 | 0 |
| rest stiffness 1 | 0 | 0 | 1 |
| postural stiffness 1 | 0 | 0 | 0 |
| akinesia 1 | 0 | 1 | 1 |
| bradikinesia 1 | 0 | 0 | 0 |
| rest waves 1 | 1 | 0 | 0 |
| postural waves 1 | 1 | 0 | 0 |
| *Weakness 0.5 | 0.5 | 0.5 | 0 |
| *Micrographia 0.5 | 0.5 | 0.5 | 0.5 |
| Combined Score | 2 | 1 | 3.5 |
| Possibly PD if score >= 3 | No | No | Yes |

*Condition: In order to count weakness and micrographia, we need at least 2 points from rest or postural tremor, rest or postural stiffness akinesia, or bradikinesia.

After the diagnosis of PD was made using the present methodology, medical history can be checked in order to rule out for example rheumatoid, arthritis type of stiffness, etc.

Table 4 illustrates a method to calculate a compound score based on objective criteria enabling us to correctly recognize all PD patients in a test population. PD is identified when the compound score >=3 (So 3.5 or higher). (*) Count weakness and micrographia only if resting and postural tremor, resting and postural stiffness, akinesia, and bradykinesia have weight 2 or higher.

TABLE 4

| Category | Condition | Data | Criterion | Point |
|---|---|---|---|---|
| 1. Tremor | 1. Resting | EMG | Significant spectral peak at 3-6 Hz | 1 |
| | 2. Postural | EMG | Significant spectral peak at 3-6 Hz | 1 |
| 2. Correlation | 1. Resting | EMG | Periodic “wave” like structure as | 1 |
| | 2. Postural | EMG | a function of the difference of | 1 |
| | | | two time intervals. At least two | |
| | | | waves should appear in correlations | |
| | | | on each side of the main diagonal | |
| | | | to count as waves. | |
| 3. Stiffness | 1. Resting | EMG | Spectral density maximum >0.5 mV | 1 |
| | 2. Postural | EMG | Spectral density maximum >1 mV in the | 1 |
| | | | writing hand or >0.5 mV in the resting hand. | |
| 4. Weakness | 2. Action | EMG | Mean intensity <0.005 mV² | 0.5 (*) |
| 5a. Slowness | 2. Action | Handwriting | Reaction Time to begin writing “3” >1 sec | 1 |
| (Akinesia) | | | | |
| 5b. Slowness | 2. Action | Handwriting | Movement Time to write “3” >1 sec | 1 |
| (Bradykinesia) | | | | |
| 6. Micrographia | 2. Action | Handwriting | Height of “cow” <7 mm | 0.5 (*) |

Two examples are presented below. The first participant has unilateral tremor at rest; both at rest and postural stiffness; correlogram periodicities at rest, which are suppressed during action; unilateral weakness, and micrographia.

Table 5: PD patient (Male; 72 years; right-handed). Compound score is 6. Therefore, this person has significant PD indicators

(*) Include both weakness and micrographia because resting and postural tremor, resting and postural stiffness, akinesia, and bradykinesia have weight 2 or higher.

TABLE 5

| | Experimental | | | PD Sub | |
|---|---|---|---|---|---|
| Symptom | Condition | Criterion | Measurement | Score | Point |
| 1. Tremor | Rest | Significant | Ch1 | Yes | 1 |
| | | spectral peak | Ch2 | Yes | |
| | | at 3-6 Hz | Ch3 | No | |
| | | | Ch4 | No | |
| | Postural | Significant | Ch1 | Yes | 1 |
| | | spectral peak | Ch2 | No | |
| | | at 3-6 Hz | Ch3 | No | |
| | | | Ch4 | No | |
| 2. Correlation | Rest | | | Yes | 1 |
| waves | Postural | | | No | 0 |
| 3. Stiffness | Rest | Average spectral | Ch1: 2 mV | Yes | 1 |
| | | density >0.5 mV | Ch2: 2 mV | Yes | |
| | | | Ch3: 1 mV | Yes | |
| | | | Ch4: 1.5 mV | Yes | |
| | Postural | Average spectral | Ch1: 3.5 mV | Yes | 1 |
| | | density >1 mV | Ch2: 7.5 mV | Yes | |
| | | for writing | Ch3: 1.9 mV | Yes | |
| | | hand Average | Ch4: 1.9 mV | Yes | |
| | | spectral density >0.5 | | | |
| | | mV for resting hand | | | |
| 4. Weakness | Action | Mean | Ch1: 0.003 mV² | Yes | 1 |
| | | intensity <0.005 | Ch2: 0.013 mV² | Yes | |
| | | mV² | Ch3: | No | |
| | | | Ch4: | No | |
| 5a. Slowness | Action | Reaction | 0.87 sec | No | 0 (*) |
| (Akinesia) | | Time >1 sec | | | |
| 5b. Slowness | Action | Writing | 1.06 sec | Yes | 0.5 (*) |
| (Bradykinesia) | | Time >1 sec | | | |
| 6. | Action | Characters | 6.4 mm | Yes | 1 |
| Micrographia | | height <7 mm | | | |

Even though we named our indicators like neurologists use in their examinations, the indicators have a different origin and are generated in an objective way. It is important that the above subject has the following positive indicators: unilateral tremor at rest that is suppressed at writing activity; stiffness at rest and during postural activity; and weakness. These are 2 out of 3 core indicators. So, if neurologists are able to observe the above symptoms during a neurological exam, they would diagnose this patient as having PD.

Table 6 shows a Control subject. A control subject is presented to illustrate that many elderly persons have various neurological or muscular disorders. However, this does not mean that they have PD.

Table 6: Control participant (Female; 44 years; right-handed). The compound score is 1. Therefore, this person has no significant PD indicators

(*) Do not include weakness or micrographia because resting and postural tremor, resting and postural stiffness, akinesia, and bradykinesia have weight less than 2.

TABLE 6

| | Experimental | | | PD Sub | |
|---|---|---|---|---|---|
| Symptoms | Condition | Criterion | Measurement | Score | Point |
| 1. Tremor | Rest | Significant | Ch1 | No | 0 |
| | | spectral peak at | Ch2 | No | |
| | | 3-6 Hz | Ch3 | No | |
| | | | Ch4 | No | |
| | Postural | Significant | Ch1 | No | 0 |
| | | spectral peak at | Ch2 | No | |
| | | 3-6 Hz | Ch3 | No | |
| | | | Ch4 | No | |
| 2. Correlation | Rest | | | No | 0 |
| waves | Postural | | | No | 0 |
| 3. Stiffness | Rest | Average | Ch1: 0.2 mV | No | 0 |
| | | spectral | Ch2: 0.2 mV | No | |
| | | density >0.5 mV | Ch3: 0.2 mV | No | |
| | | | Ch4: 0.2 mV | No | |
| | Postural | Average | Ch1: 1.8 mV | Yes | 1 |
| | | spectral | Ch2: 0.8 mV | No | |
| | | density >1 mV | Ch3: 0.2 mV | No | |
| | | for writing | Ch4: 0.2 mV | No | |
| | | hand | | | |
| | | Average | | | |
| | | spectral | | | |
| | | density >0.5 mV for | | | |
| | | resting hand | | | |
| 4. Weakness | Action | Mean | Ch1: 0.001 mV² | Yes | 0.5 |
| | | intensity <0.005 | Ch2: 0.0004 | No | |
| | | mV² | mV² | No | |
| | | | Ch3: | No | |
| | | | Ch4: | | |
| 5. Slowness | Action | Reaction | 0.80 sec | No | 0 |
| | | Time >1 sec | | | |
| | Action | Writing | 0.80 sec | No | 0 |
| | | Time >1 sec | | | |
| 6. Micrographia | Action | Character | 6.2 mm | Yes | 0.5 |
| | | height <7 mm | | | |

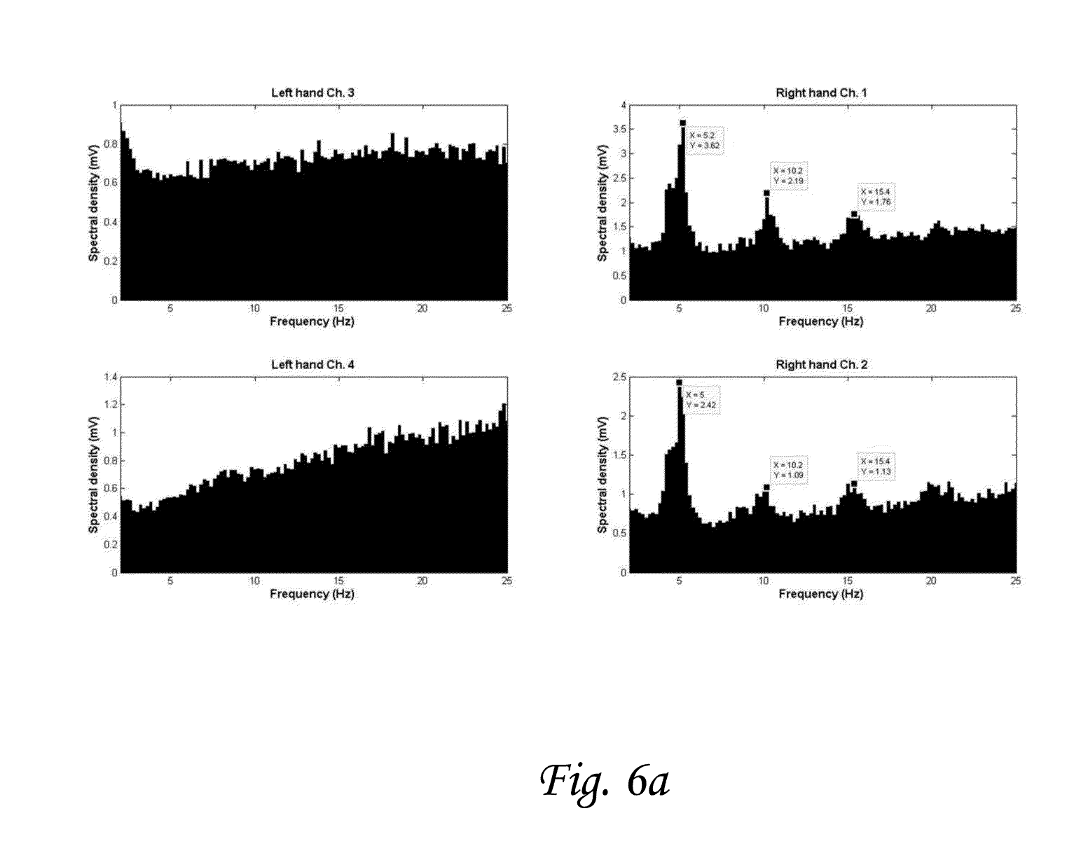

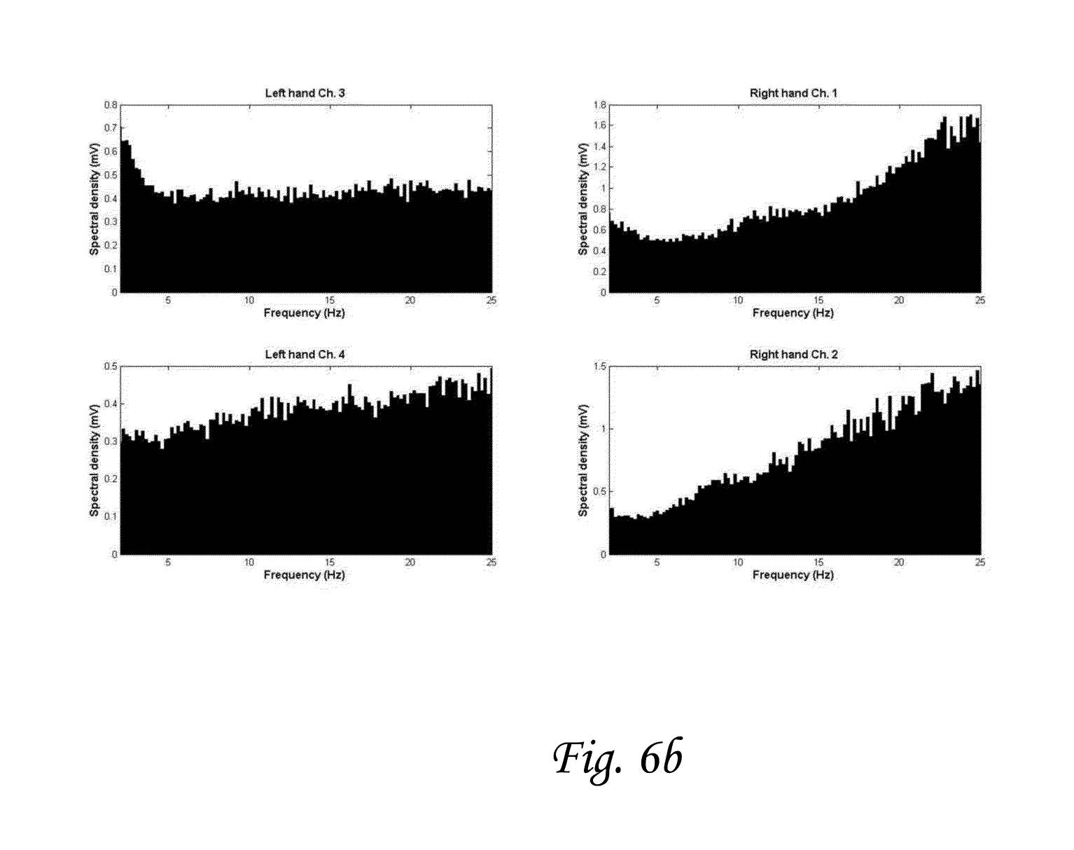

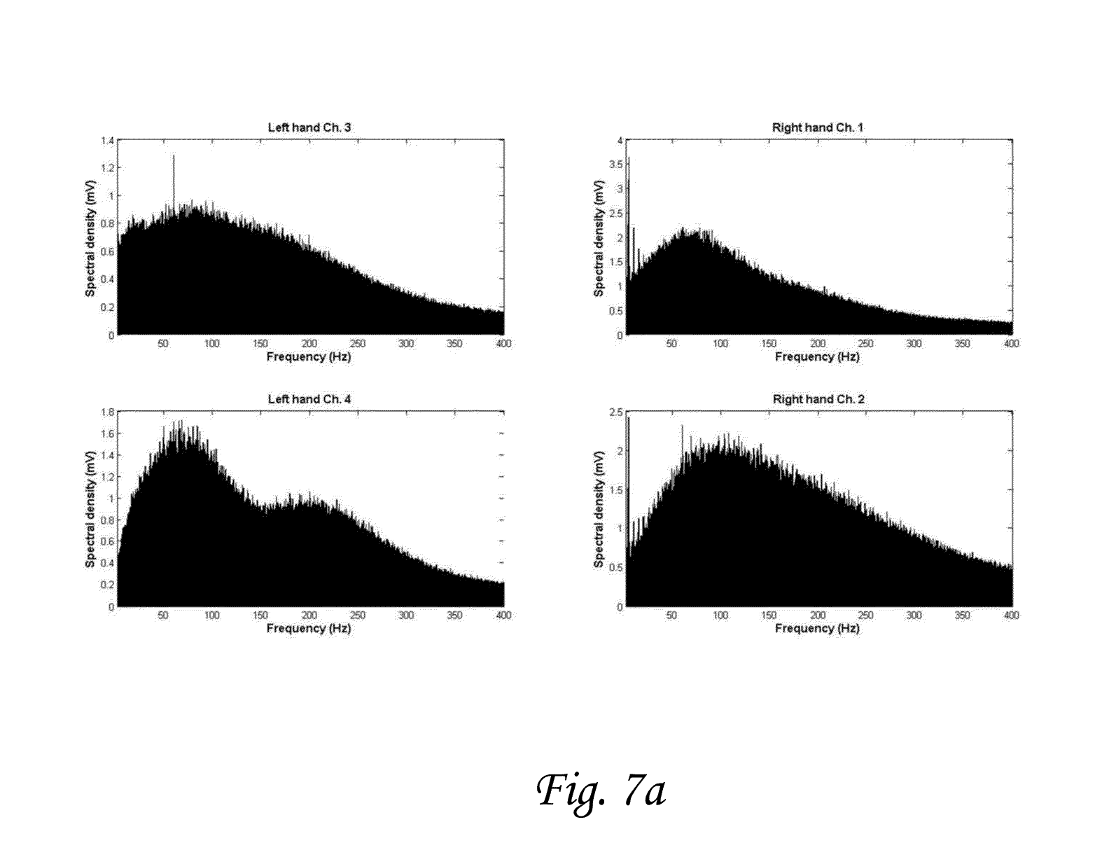

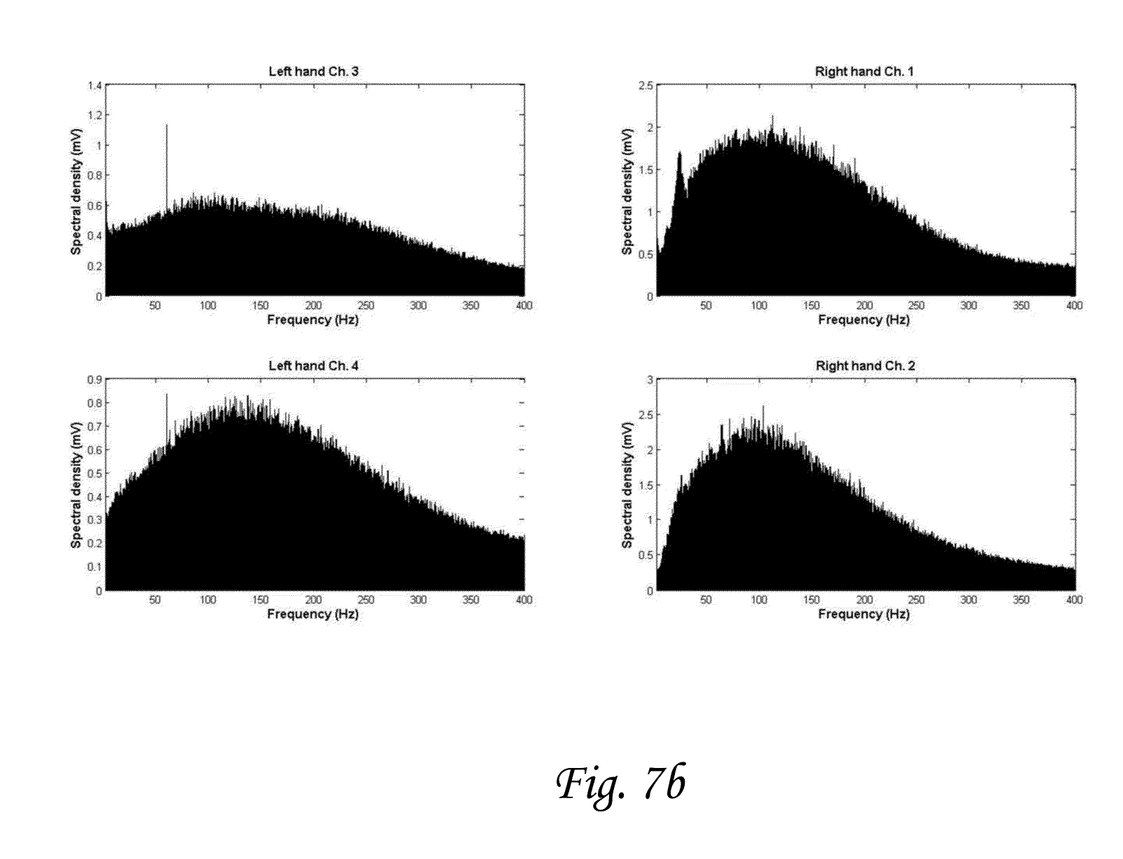

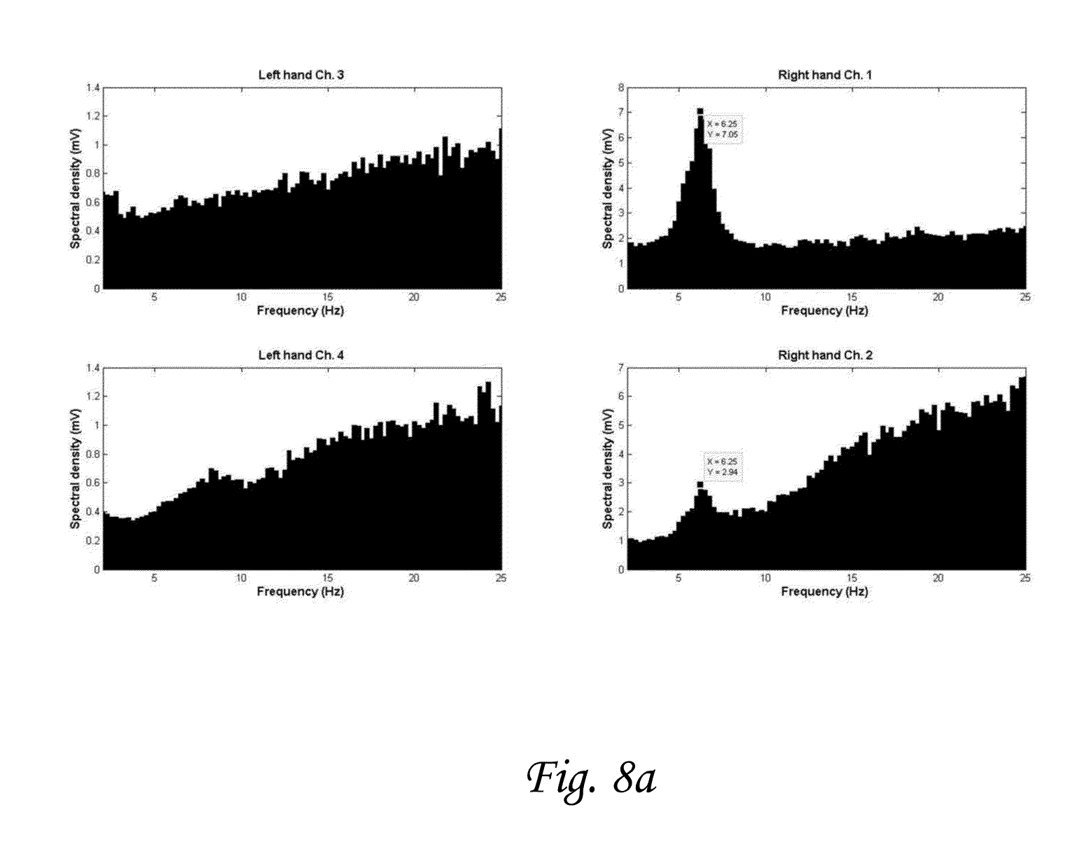

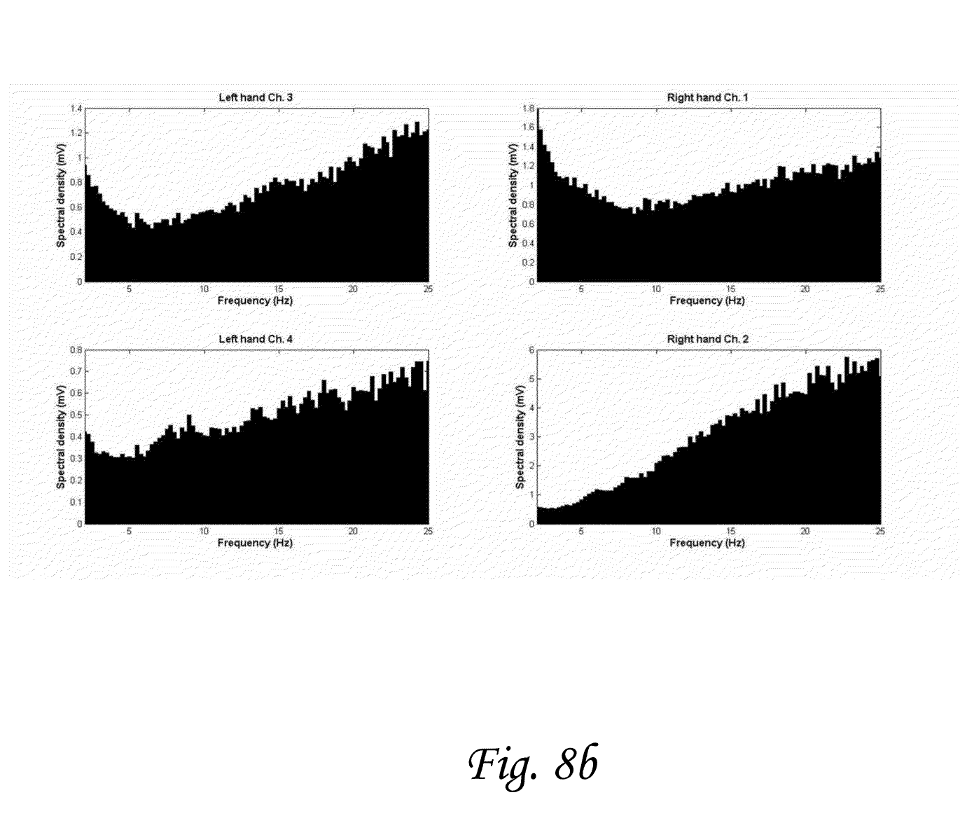

In another variant, a system and method for detection of diseases of the nervous system measures the effectiveness of medicine administered to treat the disease. EMG measurements and a score may be computed before and after administration of medicine. The following table 7 shows a result of monitoring of drug effectiveness. The individual EMG parameters are compared in the same subject before and after medication. FIGS. 4a, 5a, 6a, 7a, and 8a illustrate measurements before administration of medicine for, respectively, stiffness during postural, weakness, tremor during rest, stiffness during rest, and tremor during postural. FIGS. 4b, 5b, 6b, 7b, and 8b illustrate measurements after administration of medicine for, respectively, stiffness during postural, weakness, tremor during rest, stiffness during rest, and tremor during postural.

TABLE 7

| | | Before | After meds |
|---|---|---|---|
| EMG analysis | | meds | (Rosageline) |
| 1. Tremor | | | |
| | Rest | Yes (in 4 | No (in 4 |
| | | channels) | channels) |
| | Postural | Yes (in 4 | No (in 4 |
| | | channels) | channels) |
| 2. Stiffness | | | |
| spectral density | Rest | ch1: 2 | ch1: 2 |
| maximum >0.5 mV | | ch2: 2 | ch2: 2.4 |
| | | ch3: 1 | ch3: 0.6 |
| | | ch4: 1.5 (mV) | ch4: 0.6 (mV) |
| spectral density | Postural | ch1: 3.5 | ch1: 2 |
| maximum >1 mV | | ch2: 7.5 | ch2: 7.5 |
| | | ch3: 1.9 | ch.3: 2 |
| | | ch4: 1.9 (mV) | ch4: 1.1 (mV) |
| 3. Weakness | Writing | ch1: 0.003 | ch1: 0.003 |
| mean intensity <0.005 | | ch2: 0.013 (mV²) | ch2: 0.018 (mV²) |
| mV² | | | |

### APPENDIX

Appended hereto, and forming part of the disclosure hereof, are the following:
(A) Manuscript: 39 page paper entitled Parkinson's Disease Biomarkers Based on Electromyography and Handwriting Kinematics by Michael Linderman, Department of Biomedical Engineering, Norconnect, Inc., Ogdensburg, N.Y. 13669, USA

What is claimed is:

1. A method of detecting a disorder of the central nervous system, comprising:
recording an EMG from muscle groups on left and right hands of patient;
separating a gesture writing action into time intervals of postural and writing activity;
recording EMG activity from the same muscle groups during a resting time period;
determining a plurality of indicators, comprising:
identifying tremor peaks in EMG spectral density from more than one muscle group in each hand using separate time intervals;
determining muscle tone like stiffness from EMG spectral density and in more than one muscle group using the separate time intervals;
determining muscle weakness from EMG intensities in each time phase in more than one of the muscle group using the separate time intervals;
determining patterns of correlation waves in more than one of the muscle groups using the separate time intervals;
determining a delay when a subject started writing after hearing an audio signal;
determining the time of writing activity, when a pen is touching a tablet;
determining the heights and shapes of characters from pen traces on a tablet; and
assigning a point value to every indicator and calculating a combined score based on the point values.

2. A method of detecting a disorder of the central nervous system, comprising:
recording an EMG from muscle groups on left and right hands of a patient over time intervals, while the patient engages in writing activity;
dividing the time intervals into periods of postural and writing activity;
recording EMG activity from the same muscle groups during a resting time period;
determining a plurality of indicators, comprising:
identifying tremor peaks in EMG spectral density from more than one muscle groups in each hand using separate time intervals;
determining muscle tone like stiffness from EMG spectral density and in more than one muscle groups using the separate time intervals;
determining muscle weakness from EMG intensities in each time phase in more than one of the muscle groups using the separate time intervals;
determining patterns of correlation waves in more than one of the muscle groups using the separate time intervals; and
assigning a point value to every indicator and calculating a score based on the point values.

3. The method of detecting a disorder of the central nervous system of claim 2, wherein the EMG is measured from two channels in each hand.

4. The method of detecting a disorder of the central nervous system of claim 3, wherein the disorder is Parkinson's disease.

5. A method of detecting a disorder of the central nervous system, comprising:
recording an EMG from muscle groups on left and right hands of a patient over time intervals, while the patient engages in writing activity;
dividing the time intervals into periods of postural and writing activity;
recording EMG activity from the same muscle groups during a resting time period;
wherein during the writing activity or rest, the disorder exhibits any of: tremor in each of the muscle groups at rest or in postural, stiffness in any of the muscle groups of any hand during postural or at rest, and weakness in akinesia or bradykinesia.
